(19) United States
(12) Patent Application Publication
(10) Pub. No.: US 2008/0242644 A1
(43) Pub. Date: Oct. 2, 2008

(54) Factor Viia Inhibitor

(75) Inventors: Steven M. Torkelson, San Mateo, CA (US); Tomas Vojkovsky, San Francisco, CA (US)

Correspondence Address: WILSON SONSINI GOODRICH & ROSATI, 650 PAGE MILL ROAD, PALO ALTO, CA, 94304-1050, US

(73) Assignee: Pharmacyclics, Inc., Sunnyvale, CA (US)

(21) Appl. No.: 11/597,334

(22) PCT Filed: Jun. 2, 2005
(86) PCT No.: PCT/US2005/019420

§ 371 Date: Jan. 14, 2008

Related U.S. Application Data
(60) Provisional application No. 60/576,330, filed on Jun. 2, 2004.

(51) Int. Cl. A61K 31/4184; C07D 235/04; A61K 31/4436; A61K 31/60; A61P 7/02

(52) U.S. Cl. 514/165; 548/309.7; 514/394; 514/301

(57) ABSTRACT

The present invention relates to novel inhibitors of Factors VIIa, IXa, Xa, XIa, in particular Factor VIIa, pharmaceutical compositions comprising these inhibitors, and methods for using these inhibitors for treating or preventing thromboembolic disorders, cancer or rheumatoid arthritis. Processes for preparing these inhibitors are also disclosed.

### BACKGROUND OF THE INVENTION

1. Field of Invention

The present invention relates to novel inhibitors of Factor VIIa, pharmaceutical compositions comprising these inhibitors, and methods for using these inhibitors for treating or preventing thromboembolic disorders. Processes for preparing these inhibitors are also disclosed.

2. State of the Art

Thrombosis results from a complex sequence of biochemical events, known as the coagulation cascade. A triggering event in coagulation is the binding of the serine protease Factor VIIa (FVIIa), found in the circulation, to tissue factor (TF), a receptor, which is found on the surface of blood vessels after damage or inflammation. Once bound to TF, Factor VIIa catalyzes the formation of the serine protease Factor Xa, which subsequently forms the final protease in the cascade, thrombin.

The clinical manifestations of thrombosis range from acute myocardial infarction (AMI or heart attack) and unstable angina (UA), which occur in the key blood vessels of the heart (coronary vasculature) to deep vein thrombosis (DVT), which is the formation of blood clots in lower extremities and which often follows orthopedic surgery on the hip and knee, as well as general abdominal surgery and paralysis. Formation of DVT is a risk factor for the development of pulmonary embolism (PE) in which part of a blood clot formed in the lower extremities breaks off and travels to the lung where it blocks the flow of blood. The unpredictable development of PE often leads to a fatal outcome. Thrombosis can also be generalized systemically, with microclot formation occurring throughout the vascular system. This condition, known as disseminated intravascular coagulation (DIC), can be a consequence of certain viral diseases such as Ebola, certain cancers, sepsis, and rheumatoid arthritis. Severe DIC can lead to a dramatic reduction in the coagulation factors due to the excessive activation of the clotting response that may result in multiple organ failure, hemorrhage, and death.

The formation or embolization of blood clots in the blood vessels of the brain is the key event resulting in ischemic stroke. Triggering factors that lead to stroke are atrial fibrillation or abnormal rhythm of the atria of the heart and atherosclerosis followed by thrombosis in the main artery leading from the heart to the brain (carotid artery). Over 600,000 individuals suffer strokes each year in the U.S. Two-thirds of these stroke victims suffer some disability, and one-third suffer permanent and severe disability. Accordingly, there is a need for antithrombotic agents for the treatment of a variety of thrombotic conditions. The present invention fulfills this and related needs.

### SUMMARY OF THE INVENTION

In one aspect this invention is directed to a compound selected from the group consisting of compounds (a)-(k):

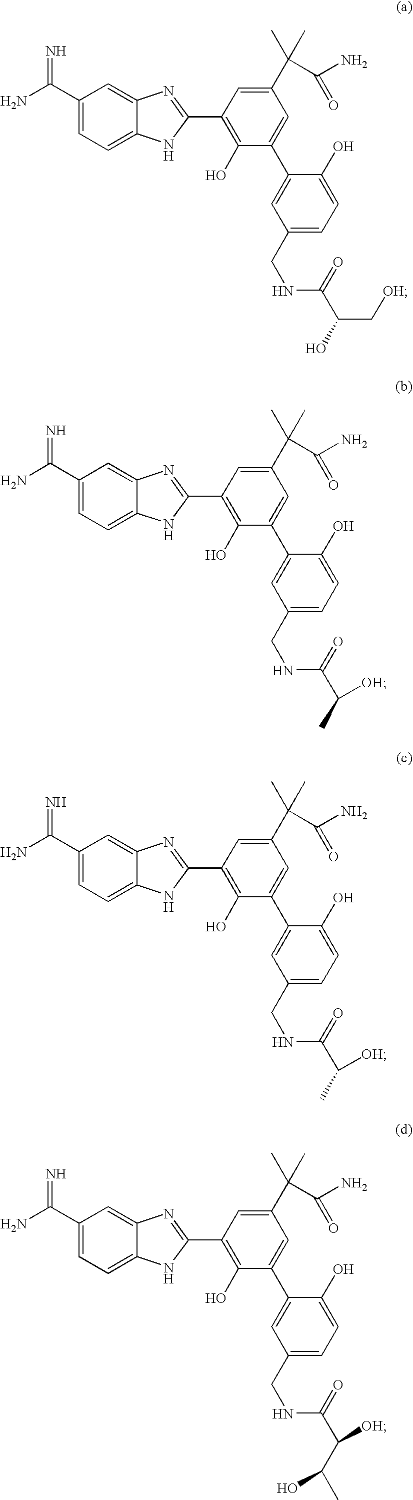

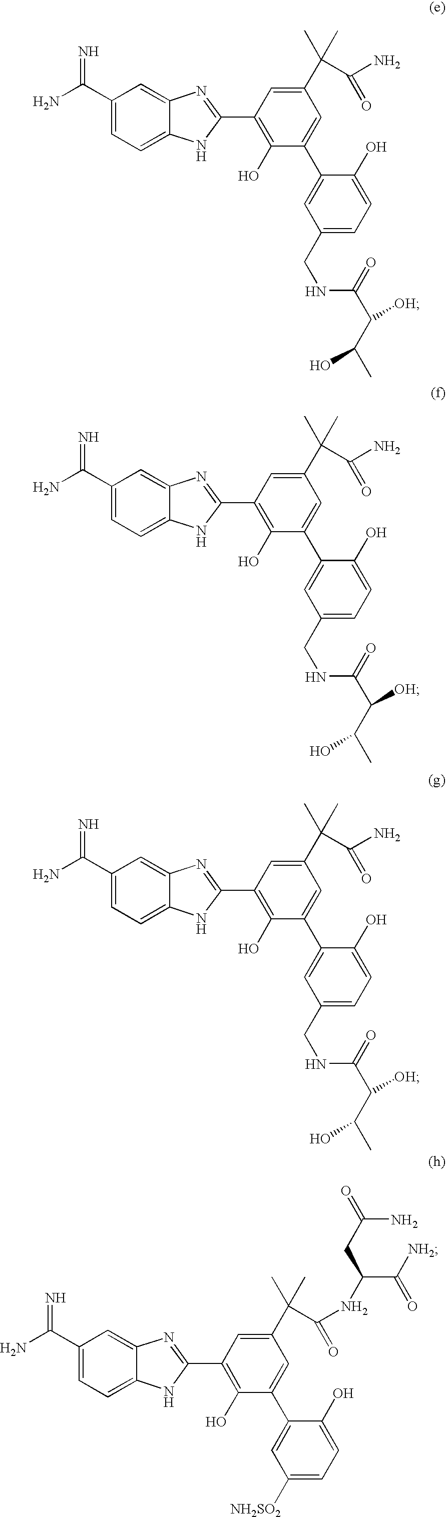

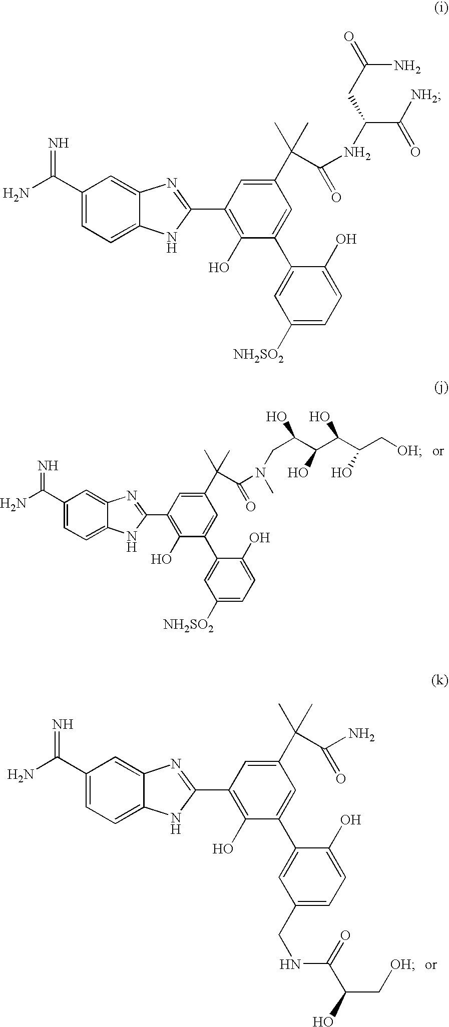

a pharmaceutically acceptable salt thereof.

In a second aspect, this invention is directed to a pharmaceutical composition comprising a pharmaceutically acceptable carrier and a therapeutically effective amount of compound (a), (b), (c), (d), (e), (f), (g), (h), (i), (j), or (k); or a pharmaceutically acceptable salt thereof.

In a third aspect, this invention is directed to a method of treating a disease in an animal that is mediated by Factors VIIa, IXa, Xa and/or XIa, preferably VIIa, which method comprises administering to said animal a therapeutically effective amount of compound (a), (b), (c), (d), (e), (f), (g), (h), (i), (j), or (k); or a pharmaceutically acceptable salt thereof. Preferably, the disorder is a thromboembolic disorder or cancer or rheumatoid arthritis, more preferably a thromboembolic disorder, even more preferably the disorder is deep vein thrombosis. Preferably, the compound of the invention is administered prophylactically.

In a fourth aspect, this invention is directed to a method of treating a thromboembolic disorder in an animal which method comprises administering to said animal a therapeutically effective amount of compound (a), (b), (c), (d), (e), (f), (g), (h), (i), (j), or (k); or a pharmaceutically acceptable salt thereof in combination with another anticoagulant agent(s) independently selected from a group consisting of a thrombin inhibitor, factor IXa inhibitor, factor Xa inhibitor, Aspirin®, and Plavix®.

In a fifth aspect, this invention is directed to a method for inhibiting the coagulation of a biological sample (e.g., stored blood products and samples) comprising the administration of compound (a), (b), (c), (d), (e), (f), (g), (h), (i), (j), or (k); or a pharmaceutically acceptable salt thereof.

In a sixth aspect, this invention directed to the use of compound (a), (b), (c), (d), (e), (f), (g), (h), (i), (j), or (k); or a pharmaceutically acceptable salt thereof in the preparation of a medicament for the treatment of a thromboembolic disorder or cancer or rheumatoid arthritis in an animal. Preferably, the disorder is a thromboembolic disorder such as deep vein thrombosis.

### DETAILED DESCRIPTION OF THE INVENTION

### Definitions

The following terms, as used in the present specification and claims, are intended to have the meanings as defined below, unless indicated otherwise.

The present invention also includes the prodrugs of compounds of the invention. The term prodrug is intended to represent covalently bonded carriers, which are capable of releasing the active compound of this invention, when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups by routine manipulation or in vivo. Prodrugs of compounds of this invention include compounds wherein a hydroxy, carbamimidoyl, amino, carboxylic, or a similar group is modified. Examples of prodrugs include, but are not limited to esters (e.g., acetate, formate, and benzoate derivatives), carbamates (e.g., N,N-dimethylaminocarbonyl) of hydroxy functional groups in compounds of the invention and the like. Prodrugs of compounds of this invention are also within the scope of this invention.

The present invention also includes (derivatives and protected derivatives of compounds of this invention. For example, when compounds of this invention contain an oxidizable nitrogen atom, the nitrogen atom can be converted to an N-oxide by methods well known in the art.

Also when compounds of this invention contain groups such as hydroxy, carboxy, carbonyl, thiol or any group containing a nitrogen atom(s), these groups can be protected with a suitable protecting groups. A comprehensive list of suitable protective groups can be found in T. W. Greene, Protective Groups in Organic Synthesis, John Wiley & Sons, Inc. 1999, the disclosure of which is incorporated herein by reference in its entirety. The protected derivatives of compounds of this invention can be prepared by methods well known in the art.

A “pharmaceutically acceptable salt” of a compound means a salt that is pharmaceutically acceptable and that possesses the desired pharmacological activity of the parent compound. Such salts include:

acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)-benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or

salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

The compounds of the present invention may have asymmetric centers. Compounds of the present invention containing an asymmetrically substituted atom may be isolated in optically active or racemic forms. It is well known in the art how to prepare optically active forms, such as by resolution of materials. All chiral, enantiomeric, diastereomeric, and racemic forms of the compounds of this invention are within the scope of this invention.

Compounds of this invention exist in tautomeric equilibrium. All possible tautomers are meant to be encompassed by such names, illustrations and descriptions and are within the scope of this invention. For example, the group —C(═NR¹³)NH₂ can tautomerize to —C(═NH)NHR¹³ group.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

“Treating” or “treatment” of a disease includes:

(1) preventing the disease, i.e. causing the clinical symptoms of the disease not to develop in a mammal that may be exposed to or predisposed to the disease but does not yet experience or display symptoms of the disease;

(2) inhibiting the disease, i.e., arresting or reducing the development of the disease or its clinical symptoms; or

(3) relieving the disease, i.e., causing regression of the disease or its clinical symptoms.

A “therapeutically effective amount” means the amount of a compound of this invention that, when administered to a mammal for treating a disease, is sufficient to effect such treatment for the disease. The “therapeutically effective amount” will vary depending on the compound, the disease and its severity and the age, weight, etc., of the mammal to be treated.

### General Synthetic Scheme

Compounds of this invention can be made by the methods depicted in the reaction schemes shown below.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure.

The starting materials and the intermediates of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., more preferably from about 0° C. to about 125° C. and most preferably at about room (or ambient) temperature, e.g., about 20° C.

Compounds of this invention can be prepared as described in Scheme I below.

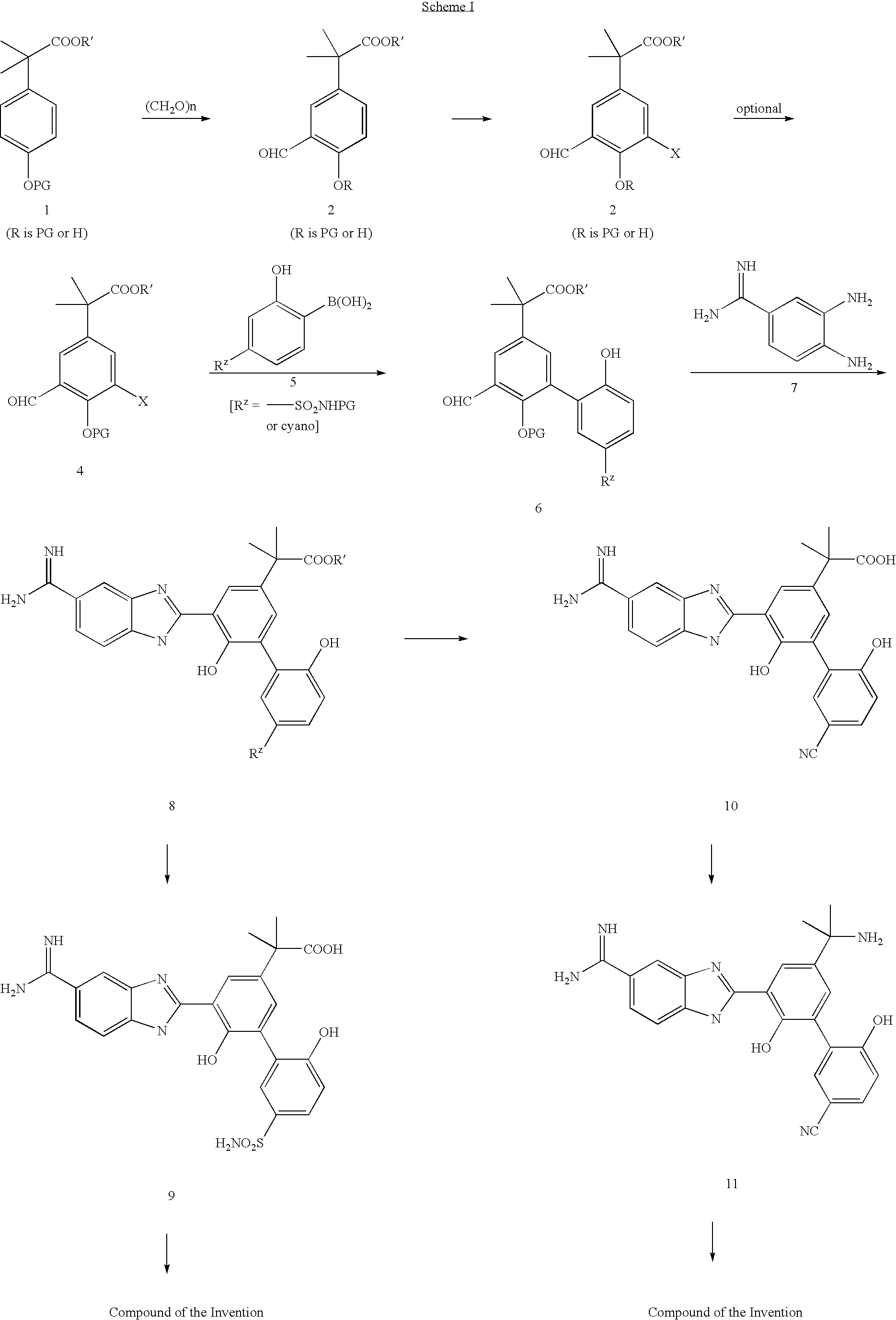

Formylation of a phenol derivative of formula 1 (where R is hydrogen or hydroxy protecting group, preferably hydroxy, and R′ is alkyl) provides a compound of formula 2. The formylation reaction is carried out in the presence of magnesium chloride and an organic base such as triethylamine, and the like, and in a suitable organic solvent such as acetonitrile, and the like. Halogenation of 2 with a suitable halogenating agent such as N-bromosuccinimide, N-iodosuccinimide, and the like and in a suitable organic solvent such as dimethylformamide, and the like provides a compound of formula 3 where X is halo.

Compounds of formula 1 can be prepared by methods well known in the art.

Protection of the hydroxy group in 3 (where R is hydrogen) with a suitable hydroxy protecting group such as alkyl, methyoxyethoxymethyl, benzyl, and the like, provides a compound of formula 4. A comprehensive list of other suitable hydroxy protective groups can be found in T. W. Greene, Protective Groups in Organic Synthesis, John Wiley & Sons, Inc. 1999, the disclosure of which is incorporated herein by reference in its entirety. Preferred hydroxy protecting group is 2-methoxyethoxymethyl and benzyl. The reaction is typically carried out in the presence of a base such as diisopropylethylamine, and the like, and in a halogenated organic solvent such as dichloromethane, carbon tetrachloride, chloroform, and the like.

Treatment of 5 with a boronic acid compound of formula 5 where R^{z} is —SO₂NHPG or cyano provides a biphenyl compound of formula 6. The reaction is carried out in the presence of a palladium catalyst such as tetrakis(triphenylphosphine)palladium and in a suitable organic solvent such as toluene or dimethoxyethane and a base such as aqueous sodium carbonate, potassium carbonate and the like. Alternatively, the reaction can be carried out in the presence of PdCl₂(dppf).CH₂Cl₂ complex in the presence of diisopropylamine in a suitable organic solvent such as tetrahydrofuran, and the like. Compounds of formula 5 they can be prepared by methods well known in the art.

Condensation of 6 with a 1,2-diamino compound of formula 7 in the presence of a suitable oxidant such as benzoquinone, air oxidation, or FeCl₃ and O₂ and in a suitable organic solvent such as methanol, ethanol, and the like, provides a compound of formula 8. Alternatively, the reaction is carried out utilizing aqueous solution of sodium metabisulfite in an alcoholic solvent such as isopropanol, and in the presence of oxygen.

Compound 8 is then converted to a compound of this invention. The procedure utilized for this conversion depends on the nature of the substituent present on the biphenyl-3-yl ring in the compound of the Invention. For example, when the substituent on the biphenyl-3-yl is —SO₂NH₂, compound 8 where R^{z} is —SO₂NHPG where PG is a suitable amino protecting group is utilized. Removal of the amino-protecting group followed by hydrolysis of the ester group provides a compound of formula 10. Compound 10 is then coupled with an amine of formula NHR^{a}R^{b} where R^{a} is hydrogen and R^{b} is (R) or (S)—CH(CONH₂)CONH₂ or R^{a} is methyl and R^{b} is R,S,S,S, —N(CH₃)CH₂CH(OH)CH(OH)CH(OH)CH₂OH provides compounds (h), (i), or (j) respectively. When the substituent on the biphenyl-3-yl ring is substituted aminomethyl group, compound 8 is first converted to a compound of formula 10 by hydrolysis of the ester group which upon reaction with ammonia provides compound II. The amination reaction is carried out reacting in the presence of a suitable coupling agent e.g., benzotriazole-1-yloxytris-pyrrolidinophosphonium hexafluorophosphate (PyBOP®), O-benzotriazol-1-yl-N,N,N′,N′-tetramethyl-uronium hexafluorophosphate (HBTU), O-(7-azabenzotriazol-1-yl)-1,1,3,3-tetramethyluronium hexafluorophosphate (HATU), 1-(3-dimethylaminopropyl)-3-ethylcarbodiimide hydrochloride (EDC), or 1,3-dicyclohexylcarbodiimide (DCC), optionally in the presence of 1-hydroxybenzotriazole (HOBT), and a base such as N,N-diisopropylethylamine, triethylamine, N-methylmorpholine, and the like. The reaction is typically carried out at 20 to 30° C., preferably at about 25° C., and requires 2 to 24 h to complete. Suitable reaction solvents are inert organic solvents such as N,N-dimethylformamide, and the like.

The cyano group is then converted to an aminomethyl group under hydrogenation reaction conditions which upon reaction with a suitable acid then provides compound (a)-(g) and (k). Detailed syntheses of compounds of this invention utilizing the above procedures are provided in working examples below.

Other methods of preparing compounds of Formula (I) are disclosed in U.S. Patent Application Hu, Huiyong et al., Publication No. 20030114457 A1 published on Jun. 19, 2003, the disclosure of which is incorporated herein by reference in its entirety.

### Utility

The compounds of this invention inhibit Factors VIIa, IXa, Xa, and XIa, in particular Factor VIIa, and are therefore useful as anticoagulants for the treatment or prevention of thromboembolic disorders in mammals.

Particular disease states which may be mentioned include the therapeutic and/or prophylactic treatment of venous thrombosis (e.g. DVT) and pulmonary embolism, arterial thrombosis (e.g. in myocardial infarction, unstable angina, thrombosis-based stroke and peripheral arterial thrombosis), and systemic embolism usually from the atrium during atrial fibrillation or from the left ventricle after transmural myocardial infarction, or caused by congestive heart failure; prophylaxis of reocclusion (i.e., thrombosis) after thrombolysis, percutaneous trans-luminal angioplasty (PTA) and coronary bypass operations; the prevention of rethrombosis after microsurgery and vascular surgery in general.

Further indications include the therapeutic and/or prophylactic treatment of disseminated intravascular coagulation caused by bacteria, multiple trauma, intoxication or any other mechanism; anticoagulant treatment when blood is in contact with foreign surfaces in the body such as vascular grafts, vascular stents, vascular catheters, mechanical and biological prosthetic valves or any other medical device; and anticoagulant treatment when blood is in contact with medical devices outside the body such as during cardiovascular surgery using a heart-lung machine or in haemodialysis; the therapeutic and/or prophylactic treatment of idiopathic and adult respiratory distress syndrome, pulmonary fibrosis following treatment with radiation or chemotherapy, septic shock, septicemia, inflammatory responses, which include, but are not limited to, edema, acute or chronic atherosclerosis such as coronary arterial disease and the formation of atherosclerotic plaques, cerebral arterial disease, cerebral infarction, cerebral thrombosis, cerebral embolism, peripheral arterial disease, ischaemia, angina (including unstable angina), reperfusion damage, restenosis after percutaneous trans-luminal angioplasty (PTA) and coronary artery bypass surgery.

The compounds of this invention can also be used in the treatment of cancer or rheumatoid arthritis.

### Testing

The ability of the compounds of this invention to inhibit factor VIIa and Xa can be tested in vitro and in vivo assays described in biological assays Example 1 and 2 below.

### Administration and Pharmaceutical Compositions

In general, the compounds of this invention will be administered in a therapeutically effective amount by any of the accepted modes of administration for agents that serve similar utilities. The actual amount of the compound of this invention, i.e., the active ingredient, will depend upon numerous factors such as the severity of the disease to be treated, the age and relative health of the subject, the potency of the compound used, the route and form of administration, and other factors.

Therapeutically effective amounts of compounds of this invention may range from approximately 0.01-50 mg per kilogram body weight of the recipient per day; preferably about 0.1-20 mg/kg/day, even more preferably about 0.25 mg/kg/day to 10 mg/kg/day. Thus, for administration to a 70 kg person, the dosage range would most preferably be about 7 mg to 1.4 g per day.

In general, compounds of this invention will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral or parenteral using a convenient daily dosage regimen, which can be adjusted according to the degree of affliction. Oral compositions can take the form of tablets, pills, capsules, semisolids, powders, sustained release formulations, solutions, suspensions, elixirs, aerosols, or any other appropriate compositions.

The choice of formulation depends on various factors such as the mode of drug administration (e.g., for oral administration, formulations in the form of tablets, pills or capsules are preferred) and the bioavailability of the drug substance. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of this invention in combination with at least one pharmaceutically acceptable excipient. Acceptable excipients are non-toxic, aid administration, and do not adversely affect the therapeutic benefit of the compound of this invention. Such excipient may be any solid, liquid, semi-solid or, in the case of an aerosol composition, gaseous excipient that is generally available to one skilled in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this invention in aerosol form. Inert gases suitable for this purpose are nitrogen, carbon dioxide, etc.

Other suitable pharmaceutical excipients and their formulations are described in Remington's Pharmaceutical Sciences, edited by E. W. Martin (Mack Publishing Company, 18th ed., 1990).

The amount of the compound in a formulation can vary within the full range employed by those skilled in the art. Typically, the formulation will contain, on a weight percent (wt %) basis, from about 0.01-99.99 wt % of a compound of this invention based on the total formulation, with the balance being one or more suitable pharmaceutical excipients. Preferably, the compound is present at a level of about 1-80 wt %. Representative pharmaceutical formulations containing a compound of this invention are described below.

The compounds of this invention can be administered alone or in combination with other compounds of this invention or in combination with one or more other active ingredient(s). For example, a compound of this invention can be administered in combination with another anticoagulant agent(s) independently selected from a group consisting of a thrombin inhibitor, a factor IXa, and a factor Xa inhibitor. Preferably, the thrombin inhibitor is Inogatran®, Melagatran® or prodrugs thereof which are disclosed in PCT Application Publication Nos. WO 94/29336 and WO 97/23499, the disclosures of which are incorporated herein by reference in their entirety. Factor Xa inhibitors that may be used in the combination products according to the invention include those described in Current Opinion in Therapeutic Patents, 1993, 1173-1179 and in international patent applications WO 00/20416, WO 00/12479, WO 00/09480, WO 00/08005, WO 99/64392, WO 99/62904, WO 99/57096, WO 99/52895, WO 99/50263, WO 99/50257, WO 99/50255, WO 99/50254, WO 99/48870, WO 99/47503, WO 99/42462, WO 99/42439, WO 99/40075, WO 99/37304, WO 99/36428, WO 99/33805, WO 99/33800, WO 99/32477, WO 99/32454, WO 99/31092, WID 99/26941, WO 99/26933, WO 99/26932, WO 99/26919, WO 99/26918, WO 99/25720, WO 99/16751, WO 99/16747, WO 99/12935, WO 99/12903, WO 99/11658, WO 99/11617, WO 99/10316, WO 99/07732, WO 9/07731, WO 99/05124, WO 99/00356, WO 99/00128, WO 99/00127, WO 99/00126, WO 9/00121, WO 98/57951, WO 98/57937, WO 98/57934, WO 98/54164, WO 98/46591, WO 98/31661, WO 98/28282, WO 98/28269, WO 98/25611, WO 98/24784, WO 98/22483, WO 98/16547, WO 98/16525, WO 98/16524, WO 98/16523, WO 98/15547, WO 98/11094, WO 98/07725, WO 98/06694, WO 98/01428, WO 7/48706, WO 97/46576, WO 97/46523, WO 97/38984, WO 97/30971, WO 97/30073, WO 97/29067, WO 97/24118, WO 97/23212, WO 97/21437, WO 97/08165, WO 97/05161, WO 96/40744, WO 96/40743, WO 96/40679, WO 96/40100, WO 96/38421, WO 96/28427, WO 96/19493, WO 96/16940, WO 95/28420, WO 94/13693, WO 00/24718, WO 99/55355, WO 99/51571, WO 99/40072, WO 99/26926, WO 98/51684, WO 97/48706, WO 97/24135, WO 97/11693, WO 00/01704, WO 00/71493, WO 00/71507, WO 00/71508, WO 00/71509, WO 00/71511, WO 00/71512, WO 00/71515, WO 00/71516, WO 00/13707, WO 00/31068, WO 00/32590, WO 00/33844, WO 00/35859, WO 00/35886, WO 00/38683, WO 00/39087, WO 00/39092, WO 00/39102, WO 00/39108, WO 00/39111, WO 00/39117, WO 00/39118, WO 00/39131, WO 00/40548, WO 00/40571, WO 00/40583, WO 00/40601, WO 00/47207, WO 00/47553, WO 00/47554, WO 00/47563, WO 00/47578, WO 00/51989, WO 00/53264, WO 00/59876, WO 00/59902, WO 00/71510, WO 00/76970, WO 00/76971, WO 00/78747, WO 01/02356, WO 01/02397, WO 01/05784, WO 01/09093, WO 01/12600, WO 01/19788, WO 01/19795, WO 01/19798, WO 93/15756, WO 94/17817, WO 95/29189, WO 96/18644, WO 96/20689, WO 96/39380, WO 97/22712, WO 97/36580, WO 97/36865, WO 97/48687, WO 98/09987, WO 98/46626, WO 98/46627, WO 98/46628, WO 98/54132, WO 99/07730, WO 99/33458, WO 99/37643 and WO 99/64446; in U.S. Pat. Nos. 6,034,093, 6,020,357, 5,994,375, 5,886,191, 5,849,519, 5,783,421, 5,731,315, 5,721,214, 5,693,641, 5,633,381, 5,612,378, 6,034,127, 5,670,479, 5,658,939, 5,658,930, 5,656,645, 5,656,600, 5,639,739, 5,741,819, 6,057,342, 6,060,491, 6,080,767, 6,087,487, 6,140,351, 6,395,731, and 5,646,165; in Japanese patent applications Nos. JP 99152269, JP 10017549, JP 10001467, JP 98017549, JP 00178243, JP 11140040, JP 12143623, JP 12204081, JP 12302765, JP 6327488 and JP 98001467; in European patent applications EP 937 723, EP 937 711, EP 874 629, EP 842 941, EP 728 758, EP 540 051, EP 419 099, EP 686 642, EP 1 016 663 and EP 529 715; and in German patent applications Nos. DE 19845153, DE 19835950, DE 19743435, DE 19829964, DE 19834751, DE 19839499, DE19900355, DE19900471 and DE 19530996, the specific and generic disclosures in all of which documents are hereby incorporated by reference.

Factor Xa inhibitors also include those disclosed in international patent applications WO 96/10022, WO 97/28129, WO 97/29104, WO 98/21188, WO 99/06371, WO 99/57099, WO 99/57112, WO 00/47573, WO 00/78749, WO 99/09027 and WO 99/57113, the specific and generic disclosures in all of which documents are hereby incorporated by reference, as well as 4-{4-[4-(5-chloroindol-2-ylsulfonyl) piperazine-1-carbonyl]phenyl}-pyridine-1-oxide and pharmaceutically acceptable derivatives thereof. Preferred Factor Xa inhibitors include antistatin, tick anticoagulant protein and those known as SQ-311 and SQ-315 (see international patent application WO 98/57951); SN-292 (see international patent application WO 98/28282); SN-429 and SN 116 (see international patent application WO 98/28269); RPR-208707 (see international patent application WO 98/25611 at Example 48); XU-817 (see international patent application WO 98/01428); SF-324 and SF-303 (see international patent application WO 97/23212); YM 60828 (see international patent application WO 96/16940 at Example 75); FACTOREX (see U.S. Pat. No. 5,783,421); SF-324 (see European patent application EP 874 629); DX9065A (see European patent application EP 540 051 at Example 39); 1-(4-carbamimidoylbenzyl)-4-(6-chloronaphthalene-2-ylsulfonyl)-piperazin-2-one (see JP 12204081 at Example 2); M55555 (see international patent application WO 99/33805 at Example 39); DPC423 (1-(3-carbamimidoylphenyl)-2-(2′-amino]sulfonyl[1, 1′-biphenyl]-4-ylaminocarbonyl)-4-bromopyrrole, see international patent application WO 98/28269); 3-(3,5-difluoro-6-[3-(4,5-dihydro-1-methyl-imidazol-2-yl)-phenoxy]-4-[2,3-dihydroxy-propoxy]-pyridin-2-yloxy)-4-hydroxy-benzamidine (see international patent application WO 00/31068); ZK-807834 (see international patent application WO 7/29067); 1,4-diaza-4-(6-chloronaphthalene-2-ylsulfonyl)-6-(methoxymethyl)-7-oxa-1′-(pyridin-4-yl)-spiro[b]cyclo-[4-3.0]-nonane-8,4′-piperidine]-2-one (see international patent application WO 01/02397); (S)-1-(4-aminoquinazolin-7-ylmethyl)-4-[2-(5-chlorothien-2-yloxy)-acetyl]-3-methoxy-methylpiperazin-2-one (see international patent application WO 00/32590); 3-(2-[4-(2-aminosulfonyl-phenyl)benzoylphenoxy)-benzamidine (see international patent application WO 01/19788); and 4-(2-[4-(5-chloroindol-2-yl-sulfonyl)-2-(pyrrolidin-1-yl-carbonylmethyl)piperazin-1-yl-carbonyl]-thiazol-5-yl)pyridine N-oxide (see Japanese patent application No. JP 12143623); as well as the compounds of Example 7 of international patent application WO 98/21188, of Examples 3 and 6 of WO 99/57113, of Example 6 of international patent application WO 00/78747, of Examples 188, 211 and 167 of U.S. Pat. No. 6,080,767, of Examples 40, 54 and 55 of international patent application WO 99/33805, of Examples 5, 6, 8, 9, 10, 11, 12, 13, 15, 16 and 17 of international patent application WO 01/05784, of Examples 6, 7, 8, 9, 10, 11, 12, 13, 14, 15, 16, 17, 22, 23, 25, 26, 28, 29, 30, 31, 32, 33, 34, 38, 39, 40, 41, 42 and 43 of international patent application WO 01/12600, and of Examples 802 and 877 of international patent application WO 00/35886. Other anticoagulant agents that can be used in the combination therapy are those disclosed in U.S. Patent Applications Publication Nos. 20020065303, 20020061842, 20020058677, 20020058657, 20020055522, 20020055469, 20020052368, 20020040144, 20020035109, 20020032223, 20020028820, 20020025963, 20020019395, 20020019394,20020016326, 20020013314, 20020002183, 20010046974, 20010044537, 20010044536, 20010025108, 20010023292, 20010023291, 20010021775, 20010020020033, 20010018423, 20010018414, and 20010000179, which are incorporated herein by reference in their entirety.

Suitable formulations for use in administering melagatran and derivatives (including prodrugs) thereof are described in the literature, for example as described in inter alia international patent applications WO 94/29336, WO 96/14084, WO 96/16671, WO 97/23499, WO 97/39770, WO 97/45138, WO 98/16252, WO 99/27912, WO 99/27913, WO 00/12043 and WO 00/13671, the disclosures in which documents are hereby incorporated by reference.

Similarly, suitable formulations for use in administering Factor Xa inhibitors and derivatives (including prodrugs) thereof are described in the literature, for example as described in the prior art documents relating to Factor Xa inhibitors that are mentioned hereinbefore, the disclosures in which documents are hereby incorporated by reference. Otherwise, the preparation of suitable formulations, and in particular combined preparations including both melagatran/derivative and Factor Xa inhibitor/derivative may be achieved non-inventively by the skilled person using routine techniques. The amounts of melagatran, Factor Xa inhibitor, or derivative of either, in the respective formulation(s) will depend on the severity of the condition, and on the patient to be treated, as well as the compound(s) which is/are employed, but may be determined non-inventively by the skilled person.

Suitable doses of melagatran, Factor Xa inhibitors and derivatives of either, in the therapeutic and/or prophylactic treatment of mammalian, especially human, patients may be determined routinely by the medical practitioner or other skilled person, and include the respective doses discussed in the prior art documents relating to melagatran (or derivatives (including prodrugs) thereof), and to Factor Xa inhibitors, that are mentioned hereinbefore, the disclosures in which documents are hereby incorporated by reference.

### EXAMPLES

All solvents and reagents were purchased from Aldrich and used as received except where noted. All reactions and products were analyzed using HPLC, employing an Agilent HP 1100 system fitted with a diode array detector and a Phenomenex Prodigy 5μ ODS-3 100A column, 150 mm×3.0 mm ID [Phenomenex catalogue #00D4096-Y0]. Chromatographic runs were performed at column temperatures of 40° C. and compound detection was performed at both 214 and 254 nm. Gradient elution was employed, using acetonitrile-water mobile phase systems with TFA as acid buffer, typically over 5-10 minute gradients.

### Reference A

### Synthesis of 3,4-diaminobenzamidine monohydrochloride

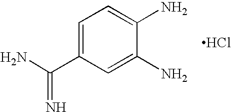

### Step 1

A mixture of 4-amino-3-nitrobenzonitrile (63.3 g, 388 mmol) in 1,4-dioxane (600 mL) and anhydrous ethanol (600 mL) was cooled in an ice water bath to 0-5° C. and treated with gaseous HCl for 1.5 h. The reaction mixture was tightly sealed and allowed to warm up to room temperature with stirring for 18 h. The flask was then carefully unsealed and the reaction mixture was diluted with anhydrous diethyl ether (about 2.4 L) until a cloudy solution was obtained. A minimum amount of absolute ethanol required to give a clear solution was then added, and the resulting solution stirred until crystals of 4-amino-3-nitro-benzimidic acid ethyl ester were observed. Ether was then cautiously added to complete the crystallization process and the suspension was allowed to stand for about 30 minutes. The crystals were filtered and washed with dry diethyl ether, then allowed to dry under aspirator vacuum. The crystals were dried in vacuo to give 4-amino-3-nitro-benzimidic acid ethyl ester hydrochloride (84.6 g) as off-white crystals.

### Step 2

4-Amino-3-nitro-benzimidic acid ethyl ester hydrochloride (84.5 g, 344 mmol) was suspended in absolute ethanol (750 mL) and then cooled to 0° C. Ammonia was then passed through the solution for a period of 2 h. The flask was tightly sealed and allowed to warm up to room temperature over an 18 h period with stirring. The product was crystallized with diethyl ether, employing a process similar to that described in Step 3 above, and the resulting solid was filtered, washed and dried to give 4-amino-3-nitrobenzamidine monohydrochloride (70.7 g) as an off-white powder.

### Step 3

A suspension of 4-amino-3-nitrobenzamidine monohydrochloride (15 g, 69 mmol) and Pearlman's catalyst [Pd(OH)₂, 1.0 g, 7.12 mmol) in methanol (200 mL) was shaken under hydrogen atmosphere 50 psi for 1.5 h. The suspension was filtered through Celite and the filtrate was added dropwise to anhydrous diethyl ether (400 mL) to precipitate 3,4-diaminobenzamidine monohydrochloride as a tan solid.

### Reference B

### Synthesis of N-tert-butyl 4-methoxy-5-(benzenesulfonamido)-3-boronic acid

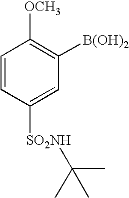

### Step

A solution of 2-iodoanisole (221.2 g, 966 mmol) in dichloromethane (2.3 L) was cooled to 0° C. and chlorosulfonic acid (64.5 mL, 112.6 g, 966 mmol) was added dropwise with stirring over a 15-minute period. The reaction mixture was allowed to warm to 10° C. over 3 h. Nitrogen gas was passed over the solution and the outlet was bubbled through a solution of aqueous sodium hydroxide to scrub the gaseous hydrogen chloride produced in the reaction. An aliquot of the reaction was analyzed by HPLC, which showed that 2-iodoanisole had been consumed. The reaction mixture was treated with phosphorus pentachloride (217.8 g, 1.045 mol) and stirred at room temperature for 2 h. The reaction mixture was concentrated in vacuo to remove most of the volatile components then further concentrated at a bath temperature of 100° C. to remove POCl₃ produced in the reaction. The resulting oily residue was dissolved in CH₂Cl₂ (2.8 L) and this solution was stirred with water (3 L) while solid sodium bicarbonate was added to maintain the pH around 7. The layers were separated and the organic phase was cooled to 0° C., then tert-butylamine (230 mL, 160 g) was added at such a rate to maintain the internal temperature ≦10° C. The reaction mixture was allowed to warm up to ambient temperature overnight, then washed with 5% sodium hydroxide. The organic phase was concentrated in vacuo to give N-tert-butyl 3-iodo-4-methoxybenzenesulfonamide (340 g) as an off-white solid.

### Step 2

N-tert-Butyl 3-iodo-4-methoxybenzenesulfonamide (335 g, 907 mmol) was dissolved in dichloromethane (3 L) and the resulting solution was cooled to an internal temperature of −20° C. The solution was treated with a 3.0 M solution of methylmagnesium bromide in diethyl ether (308 mL, 925 mmol) dropwise over 0.5 h to maintain the internal temperature of the flask at −20±5° C. The reaction mixture was allowed to stir at −20±5° C. for 2.5 h then a 2.13 M solution of isopropylmagnesium bromide in diethyl ether (511 mL, 1.09 mol) was added at ca −35° C. The resulting solution was allowed to stir at −35±5° C. for 1.5 h. The reaction mixture was warmed to 0° C. and additional isopropylmagnesium bromide in diethyl ether (86.0 mL, 183 mmol) was added. The reaction mixture was stirred for 2 h at 0° C., then an additional aliquot of isopropylmagnesium bromide in diethyl ether (25.0 mL; 53.3 mmol) was added. The reaction mixture was treated with trimethylborate (320 mL; 2.90 mol) in THF (175 mL) in one portion, resulting in a temperature increase to 27° C. The reaction mixture was stirred at this temperature for 4 h, then poured into water (1.3 L) and 85% phosphoric acid was added until the solution was pH 2. The layers were separated and the organic phase was washed with 1.5 N aqueous NaOH (2 L), followed by 1% aqueous NaOH (2 L). The combined aqueous phases were acidified with phosphoric acid to pH 2 and the resulting acidic solution was extracted with 9:1 dichloromethane/THF solution (2 L followed by 1 L). The organic phase was dried (Na₂SO₄), filtered and concentrated in vacuo to give about 250 g of a white solid which was dissolved in ethanol (1 L). The solution was diluted with water to give a total volume of 4 L and the resulting solution was stirred at room temperature overnight. The resulting crystalline solid was filtered and dried under high vacuum overnight to afford N-tert-butyl 4-methoxy-5-(benzene-sulfonamido)-3-boronic acid (221 g) as a white solid, which was a dihydrate (approximately). The filtrate was extracted with a 9:1 solution of dichloromethane/THF and the extract evaporated. The crude solid (23 g) was recrystallized from a 3:1 solution of water/ethanol (500 mL) to yield an additional 19 g of product as a white solid.

### Reference C

### Synthesis of 4-benzyloxy-N-tert-butyl-3-boronic acid-benzenesulfonamide

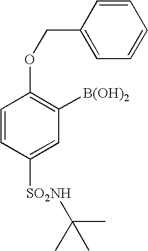

### Step 1

To a 1 L round bottom flask was added 2-iodophenol (50 g) and nitromethane (250 mL) and the reaction mixture was cooled to 0° C. Fuming sulfuric acid (42 mL, 30% SO₃) was added dropwise and the reaction mixture was allowed to warm to room temperature. After 2 h, the reaction was complete and it was poured into water (400 mL) and washed with ethyl acetate (200 mL). The organic was then back extracted with water (300 mL) and concentrated to oil and combined with the original aqueous layer. The aqueous layer was then neutralized with 5 M aqueous sodium hydroxide (300 mL) and transferred to a 2L RBF. Sodium hydroxide pellet (11 g), ethanol (150 mL), and benzyl bromide (50 mL) were then added and the reaction mixture was heated to an oil bath temperature of 82° C. and stirred for 16 h. After the reaction was complete, ethanol was removed by vacuum distillation which caused the product to precipitate out of solution. The product was then filtered and dried under high vacuum to give 4-benzyloxy-3-iodo-benzenesulfonic acid (61 g, 70% yield).

### Step 2

To a 2L RBF was added 4-benzyloxy-3-iodo-benzenesulfonic acid (49.87 g) and dichloromethane (1000 mL). The suspension was stirred and phosphorous pentachloride (53 g) was added causing the reaction to became a solution. After heating the reaction mixture at 40° C. for 1 h, aqueous sodium hydroxide (400 mL of 20%) was then slowly added and stirring was continued until the aqueous was pH 7. The organic layer was separated and stirred with 50% aqueous saturated sodium bicarbonate (125 mL) for 30 minutes (pH 10). The organic layer was separated, dried with anhydrous sodium sulfate, decanted to a 2L RBF and tert-butylamine (34 mL) was added. After 16 h, the reaction mixture was basified to pH 13-14 with 5% aqueous sodium hydroxide. The organic layer was separated and concentrated to a solid which was then slurried at 50° C. in isopropyl acetate, cooled, and filtered to give 4-benzyloxy-N-tert-butyl-3-iodo-benzenesulfonamide (46 g, 80% yield) in two crops.

### Step 3

To a 1L RBF was added 4-benzyloxy-N-tert-butyl-3-iodo-benzenesulfonamide (32 g) and dichloromethane (320 mL) and the reaction mixture was stirred and cooled to −20 to −25° C. Methyl magnesium bromide (24.4 mL, 3 M in ether) was added dropwise. The reaction mixture was stirred for 2 h and then cooled to −35 to −40° C. Isopropyl magnesium bromide (54 mL of 2.13 M in ether) was added dropwise. Tetrahydrofuran (17 mL) and trimethyl borate (6 mL) were then added precipitating a white solid and raising the internal temperature of the reaction mixture to 0° C. The reaction mixture was allowed to warm to room temperature and after 12 h phosphoric acid (250 mL of 1M in 500 mL of water) was added. The organic layer was separated and basified with 2.5% aqueous sodium hydroxide (500 mL) causing some of the product to precipitate. The aqueous layer along with some of the precipitated solids was then acidified with concentrated phosphoric acid to a pH of 2 and extracted with 10% tetrahydrofuran in dichloromethane. The solids were carried on with the organic which was then concentrated to give a white solid that was then slurried in 1 L of water for 30 minutes. The solid was filtered and dried under high vacuum to give 4-benzyloxy-N-tert-butyl-3-boronic acid-benzenesulfonamide (23 g, 88% yield).

Alternate Synthesis of the Title compound:

### Step 1

A 3-neck, 3 L—round-bottom flask was equipped with an over-head stirrer, thermometer, N₂ line, 250 mL pressure-equalizing dropping funnel, and gas-exit scrubber (to NaOH solution). The flask was flushed with N₂ and charged with commercially available 2-iodophenol (Alfa Aesar; 201.95, 0.918 mol) and dry dichloromethane (920 mL). A gentle stream of N₂ was established through the reaction head-space, the reaction vessel then immersed in a brine-ice bath and cooled to −5° C. The dropping funnel was charged with dry dichloromethane (175 mL), then chlorosulfonic acid (Aldrich; 106.96 g, 0.918 mol, 1.00 eq.), and the resulting mixture was stirred with a Teflon rod. The dilute solution of chlorosulfonic acid was then added dropwise to the reaction mixture over a period of approx. 90 mins. A thick pink slurry formed during the addition. Thirty minutes after complete addition, the ice bath was removed and the reaction mixture was allowed to stir at ambient temperature. After 2 h, the reaction vessel was immersed in a cold-water bath and water (500 mL) was added to the reaction mixture over a few minutes. The resulting mixture was stirred vigorously until it was biphasic/homogenous upon settling. The mixture was transferred to a separating funnel along with water and was extracted with dichloromethane. The aqueous layer containing 4-hydroxy-3-iodo-benzenesulfonic acid was transferred back to the original reaction vessel for the next step.

### Step 2

Sodium hydroxide (pellets, 110 g, 2.75 mol, 3.00 eq) was added portionwise to the vigorously stirring aqueous solution of the 4-hydroxy-3-iodo-benzenesulfonic acid. After addition was complete, 10-15 min., isopropyl alcohol (150 mL) was added to the resulting white suspension. The dropping-funnel was charged with benzyl bromide

(Aldrich; 164.9 g, 0.964 mol, 1.05 eq.) and added to the reaction mixture over a period of approx. 5 mins. and the reactin mixture was heated to 80°≦Tᵢₙₜ≦84° C. After approx. 25 min. it was determined that the reaction was not proceeding further and therefore additional sodium hydroxide (3.67 g, 91.8 mmol, 0.1 eq.) and then benzyl bromide (15.7 g, 91.8 mmol, 0.1 eq.) were added to the reaction mixture to give a homogenous solution. After 70 min. from the original benzyl bromide addition, the heating was stopped and the reaction was allowed to cool slowly in the oil-bath with stirring. At 7.5 h, the reaction mixture appeared as a suspension of fine-reflective precipitate in brown liquid. The reaction mixture was acidified with 3:1 water-sulfinuric acid from pH 13+ to between pH 7.5 and 8 (approx. 70 mL is required). The reaction mixture was then cooled gradually to about 5° C. and stirred at that temperature for ˜1 h. The waxy white plaques were collected by filtration, washed with dichloromethane and dried under high vacuum (lyophilizer, 100-200 mTorr) for ˜24 h to give sodium 4-benzyloxy-3-iodo-benzenesulfonate as a brilliant white, crystalline solid, (267.7 g, 71%).

### Step 3

A 3-neck, 3 L, round-bottom flask was equipped with an over-head stirrer, reflux condenser (with gas exit to NaOH scrub solution), and a pressure-equalizing dropping-funnel with N₂ line. The flask was flushed with N₂, charged with sodium 4-benzyloxy-3-iodo-benzenesulfonate (234 g, 0.568 mol), dichloromethane (1.15 L), and catalytic amount of dimethylformamide (910 mg, 11.7 mmol, 2.1 mol %). The white suspension was stirred under a gentle stream of nitrogen and heated in an oil-bath set to 40-45° C. Oxalyl chloride (90.1 g, 0.710 mol, 1.25 eq) was then added over 3-5 min. After 2.5 h, the reaction was allowed to cool to 25° C. in a cold-water bath and then quenched drop-wise with water (60 mL) over approx. 5 min. A further portion of water (450 mL) was added in a single portion and the reaction mixture stirred vigorously for 5-10 min. The organic layer was separated and washed with water until the aqueous pH had increased to pH 4 to 5). The resulting dichloromethane solution of 4-benzyloxy-3-iodo-benzenesulfonyl chloride was used in the next step.

### Step 4

A 3-neck, 3 L, round-bottom flask was equipped with an over-head stirrer, thermometer, and a pressure-equalizing dropping-funnel was charged with the solution of 4-benzyloxy-3-iodo-benzenesulfonyl chloride. The flask immersed in a cold water bath (Tᵢₙₜ=22° C.) and tert-butylamine (90.1 g, 0.710 mol, 2.1 eq) was added drop-wise (Tᵢₙₜ no change). The resulting reaction mixture was stirred overnight at the ambient water-bath temperature. After 17 h, the reaction mixture was worked-up and the organic layer was separated and concentrated to approx. ⅓ (˜500 mL) of its original volume at which point the product started to precipitate. The reaction mixture was warmed to 35-40° C. at atmospheric pressure till the solids had re-dissolved. The solution was then allowed to cool, with gentle stirring, to room temperature. Within 2 days a white precipitate had formed. The suspension was stirred vigorously while hexane (1.5 L) was slowly added, then stirred overnight, and then cooled in an ice-bath for 1-2 h The precipitate was collected by filtration and washed with hexane, dried, first under suction to give 4-benzyloxy-N-tert-butyl-3-iodo-benzenesulfonamide (238, 94%).

### Step 5

A 3-neck, 2 L-round-bottom flask was equipped with an over-head stirrer, thermometer, pressure-equalizing dropping-funnel, and an N₂ line. The flask was flushed with N₂ and then charged with 4-benzyloxy-N-tert-butyl-3-iodo-benzenesulfonamide (198.6 g, 0.446 mol) and dichloromethane (600 mL). The white suspension was stirred under a gentle stream of N₂ and cooled in an ice-water bath (0° C.≦Tᵢₙₜ≦5° C.). The dropping-funnel was charged with methyl magnesium bromide (Aldrich; 3.0 M in diethyl ether, 167 g, ˜171 mL, 0.513 mol, 1.15 eq), which was added dropwise to the suspension at such a rate so as to maintain Tᵢₙₜ<5° C. (addition of salt to the cool-bath was necessary) to give a colorless-homogenous mixture within ⅓ addition. After the addition was completer, the dropping-funnel was charged with isopropylmagnesium bromide (Boulder Scientific; 2.13 M in diethyl ether; 250 mL, 0.533 mol, 1.2 eq), which was added dropwise to the reaction mixture at such a rate so as to maintain Tᵢₙₜ<5° C. After the addition was complete, the reaction mixture was stirred for 15-20 min. The dropping-funnel was removed and replaced with a septa and cannula, and the reaction mixture was transferred over 2 h to a 3-necked, 3-L round bottom flask containing a solution trimethyl borate (106.6 g, 1.03 mol, 2.30 eq) and tetrahydrofuran (600 mL) and maintained under nitrogen atomosphere at (Tᵢₙₜ<5° C.) utilizing an ice-water bath. After the addition was complete, the solution was allowed to stir at <5° C. for 30 min. and then transferred to a separatory funnel and washed with an equal volume of a 2:1 (water:phosphoric acid) solution. The organic layer was dried over sodium sulfate. Ethyl acetate was added to the solution and the combined organic layer was concentrated to give 4-benzyloxy-N-tert-butyl-3-boronic acid-benzenesulfonamide (129 g, 80%).

### Reference D

### Synthesis of methyl 2-(3-bromo-5-formyl-4-hydroxyphenyl)-2-methylpropanoate

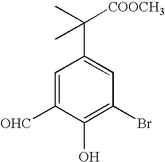

### Step 1

A 3 L, 3-neck round-bottom flask was equipped with an over-head stirrer, thermometer and a pressure-equalizing addition funnel. The flask was charged with 1.0 M tert-BuOK solution in THF (Aldrich; 1822 g, 2.020 L, 2.020 mol) and then purged with nitrogen. The solution was stirred and immersed in a cooling bath of cold tap water (internal temp. 18° C.). 4-Methoxyphenylacetonitrile (148.7 g, 1.010 mol) was added neat via the addition funnel over a period of 30 minutes. The addition funnel was washed with THF and the washings were added. The reaction was stirred for 20 minutes, then the addition funnel was charged with iodomethane (286.7 g, 2.020 mol), which was added dropwise over a 55-minute period, resulting in a milky, salmon-colored suspension. The addition rate was adjusted to maintain an internal temperature of 21-27° C. and ice was added to the cooling bath to assist in maintaining this temperature range. After the addition was complete, the reaction mixture was stirred for an additional 60 minutes, then it was poured into a mixture of saturated aqueous sodium chloride and water (2:1; 1.5 L) and the reaction vessel rinsed with portions of saturated aqueous sodium chloride (250 mL) and THF (100 mL). The combined liquids were shaken and the resulting layers were separated, then the organic phase was concentrated in vacuo. The resulting residue was dried under high vacuum overnight to give 2-(4-methoxyphenyl)-2-methylpropionitrile an orange-brown oil, containing a small amount of a white precipitate. This material was used directly in the next step without additional processing.

### Step 2

A mixture of 2-(4-methoxyphenyl)-2-methylpropionitrile (358 g, 2.04 mol), KOH (284.8 g, 5.08 mol), ethylene glycol (750 mL), and water (100 mL) was heated at 150-160° C. for 7 h in a 1 L round-bottom flask equipped with a bump flask and fermentation lock, then allowed to cool and stand overnight. Heating was continued for an additional 7 hours, without any additional conversion being observed. The reaction was allowed to cool and poured into water (2 L), then acidified with stirring to pH 10-11 by addition of concentrated HCl (˜250 mL). The resulting solution was extracted with isopropyl acetate (1×1 L, followed by 2×500 mL) and then filtered to remove a small quantity of a white precipitate. The reaction mixture was stirred vigorously and slowly acidified further to ca pH=2 with concentrated HCl (˜250 mL). The product started to precipitate at pH 6-7. The suspension was stirred for 30 minutes at ambient temperature then kept a refrigerator overnight. The mixture was filtered and the precipitate was washed with cold 1M HCl (500 mL), followed cold water (2×150 mL). The solid was dried under suction, follow by high vacuum (lyophilizer) overnight to give 2-(4-methoxyphenyl)-2-methylpropanoic acid (314 g, 80%) as pale yellow-orange crystals containing approximately 2.0% of the mono-methyl impurity.

### Step 3

A mixture of 2-(4-methoxyphenyl)-2-methylpropanoic acid (45.3 g; 233 mmol) and pyridine hydrochloride (150 g; 1.30 mol) was heated for 5 hours under nitrogen at an oil bath temperature of 180-190° C. The reaction mixture was allowed to cool to 90° C., then diluted with water (400 mL) and concentrated HCl (30 mL). The resulting solution was extracted with ethyl acetate (55 mL) and the organic layer was washed with water (5×500 mL). The combined aqueous extracts were washed with ethyl acetate (400 mL) and the combined organic phases were dried (MgSO₄) and concentrated in vacuo. The solid residue (40.9 g) was dissolved in a mixture of ethyl acetate (60 mL) and benzene (200 mL) previously heated to reflux. Hexane (100 mL) was added to the refluxing mixture and the resulting slurry was allowed to cool to room temperature overnight. The solid was filtered, washed with hexane-benzene (1:1) and dried under vacuum to give α,α-dimethyl 4-methoxyphenylacetic acid (35.70 g; 85%) as a white solid, containing approximately 2.3% of the mono-methyl impurity.

### Step 4

α,α-Dimethyl 4-methoxyphenylacetic acid (108 g; 0.556 mol) was heated with pyridine-HCl (324 g; 2.80 mol) to 180° C. for 5 hours. The reaction mixture was allowed to cool to ca 90° C., then added to an equal volume of 10% aqueous sodium hydroxide and chipped ice to give a basic solution. The aqueous solution was washed with diethyl ether, then acidified to pH=3 with 85% H₃PO₄. The aqueous phase was extracted with ethyl acetate and the combined organic extracts were concentrated in vacuo to give a solid, which was recrystallized from water to give α,α-dimethyl 4-hydroxyphenylacetic acid.

### Alternate Procedure

α,α-Dimethyl 4-methoxyphenylacetic acid (100 g; 0.515 mol) and pyridine.HCl (297 g; 2.57 mol) were heated with stirring at 180° C. for 5 hours. The reaction mixture was allowed to cool to room temperature and then stand overnight. After 18 hours, the reaction was re-heated such that it was homogeneous for the purpose of sampling and then cooled to ˜100° C. and poured onto a mixture of IL chipped-ice and 10% aqueous NaOH (1.5 L). The aqueous layer was extracted with diethyl ether, then acidified with 85% aqueous H₃PO₄ to pH=3 (˜130 mL), and then extracted with EtOAc (500 mL, followed by 250 mL). The combined organic extracts were concentrated in vacuo to give a solid, which was crystallized from hot water. Once crystals had significantly established stirring was started at a rate so as to maintain mobility of the entire precipitate and then continued overnight. The mixture was cooled in a fridge for 2 hours prior to collection of the crystals by filtration. The crystals were washed with a minimum amount of cold water (˜100 mL), and then dried, first under suction and then under high vacuum (lyophilizer) to give α,α-dimethyl 4-hydroxyphenylacetic acid (76.3 g, 82%) as a tan crystalline solid.

### Step, 5

A mixture of α,α-dimethyl 4-hydroxyphenylacetic acid (90.0 g; 499 mmol) and methanol (1 L) was cooled to 0° C. in an ice water bath. To this solution was added thionyl chloride (72.9 mL; 119 g) dropwise with stirring. The resulting solution was heated under reflux for 2 hours. The solution was cooled to room temperature and concentrated in vacuo to afford a solid, which was crystallized from toluene (900 mL) to give methyl 2-(4-hydroxyphenyl)-2-methylpropanoate (92 g, 95%).

### Alternate Procedure

Thionyl chloride (92.4 g; 777 mmol; 200 mol %) was added over 20 minutes to a stirring solution of α,α-dimethyl 4-hydroxyphenylacetic acid (70.0 g; 388 mmol) in methanol (390 mL). The resulting solution was allowed to stir at ambient temperature for 40 minutes, concentrated, and dried further under high vacuum to give methyl 2-(4-hydroxyphenyl)-2-methylpropanoate (75.3 g, 100%) as a light-brown semi-crystalline solid.

### Step 6

To a 5 L RBF with overhead stirrer, condenser, thermocouple, and heating mantle was added methyl 2-(4-hydroxyphenyl)-2-methylpropanoate (90.0 g, 0.463 mol), followed by acetonitrile (2250 mL). Triethylamine (260 mL; 189 g) was then added, followed by anhydrous magnesium chloride (88.0 g; 924 mmol). The reaction mixture was stirred for 30-45 minutes, paraformaldehyde in the form of prills (99.0 g; 3.30 mol) was added and the reaction was heated to reflux. Analysis of the reaction mixture by HPLC showed the reaction was complete after about 2 hours. The reaction was then cooled and diluted with diethyl ether (3L) and 1N aqueous HCl (3L). The layers were separated and the organic phase was washed with 1N HCl (3×3L) and saturated aqueous sodium chloride, then dried (Na₂SO₄). Concentration of the solution afforded an oil, which was crystallized by stirring with hexane in a dry ice bath to give methyl (4-hydroxy-3-formylphenyl)-2-methylpropanoate (94.7 g, 92%) as a white solid.

### Alternate Procedure

A 3-L, 3-necked round bottom flask equipped with an overhead stirrer, condenser, and thermometer was charged with methyl 2-(4-hydroxyphenyl)-2-methylpropanoate (75.3 g; 388 mmol), and acetonitrile (400 mL). With stirring, triethylamine (47.1 g; 0.466 mol; 120 mol %) was added in a single portion and then anhydrous magnesium chloride (40.6 g; 427 mmol; 110 mol %) portion-wise over 3-5 minute period. The reaction mixture was stirred at 80-82° C. for 30 minutes and then paraformaldehyde (23.3, 776 mmol; 200 mol %) added portion-wise over a 5-minute period. Within a few minutes, the reaction began to turn from a light-brown suspension to a homogeneous yellow solution. The reaction was stirred at 80-82° C. and monitored by HPLC for conversion of starting material. The reaction mixture was allowed to cool to ˜60° C., then 1M aqueous H₃PO₄ (˜100 mL) added, and the resulting dense yellow suspension concentrated in vacuo to a pasty solid. Dichloromethane (1 L) and water (1 L) was added, the mixture stirred vigorously with an overhead stirrer, and 85% aqueous H₃PO₄ added until the solids dissolved and pH=3 was attained (˜70 mL). The layers were separated and the organic layer washed with 1M H₃PO₄ (200 mL), brine (200 mL), and concentrated to give methyl (4-hydroxy-3-formylphenyl)-2-methylpropanoate (84.5 g, 98% mass yield) of an orange-brown oil that contained some unreacted starting material.

### Step 7

To a 5 L RBF with overhead stirrer and thermocouple was added methyl (4-hydroxy-3-formylphenyl)-2-methylpropanoate (121 g, 547 mmol) followed by N,N-dimethylformamide (DMF; 1600 mL). The solution was then cooled to 5° C. and a solution of N-bromosuccinimide (117 g, 657 mmol) in DMF (700 mL) was added dropwise at a rate to maintain the internal temperature below 10° C. After the addition was complete, the reaction mixture was allowed to warm to room temperature and the reaction was complete within 3 hours. The reaction mixture was diluted with diethyl ether (3L) and the resulting solutions washed with water (4×μL). The first water wash was back extracted with diethyl ether (IL) and combined for the remaining washes. The organic phase was then washed with saturated aqueous sodium chloride (2×1 L), dried (Na₂SO₄), and concentrated to give a reddish orange solid. The solid is dissolved in hot isopropanol (120 mL) and allowed to cool while stirring. The crystalline product was then filtered and washed with an equal amount of cold (−20° C.; 120 mL) IPA to yield methyl (5-bromo-4-hydroxy-3-formylphenyl)-2-methylpropanoate (132 g, 80.1%) as an off-white solid.

### Example 1

### Synthesis of N-[3′-(5-carbamimidoyl-1H-benzoimidazol-2-yl)-5′-(1-carbamoyl-1-methyl-ethyl)-6,2′-dihydroxybiphenyl-3-ylmethyl]-(2S)-2,3-dihydroxypropionamide dihydrochloride

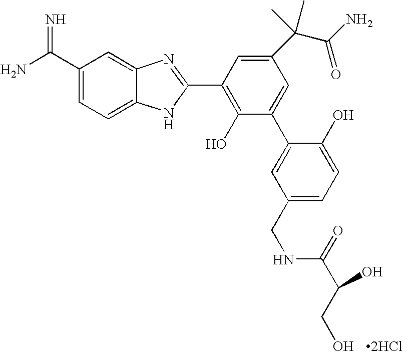

### Step 1

To a 3-L round bottom flask with a magnetic stir bar was added methyl 2-(3-bromo-5-formyl-4-hydroxyphenyl)-2-methylpropanoate (132 g, 438 mmol), potassium carbonate (66.7 g, 483 mmol) and DMF (1 L). The solution was allowed to stir at room temperature for 0.5 h. Methyl iodide (31.5 mL, 506 mmol) was added dropwise with vigorous stirring. The reaction was complete after 3 hours. To this solution, methyl tert-butyl ether (MTBE) (3 L) was added and the solution was filtered to remove the inorganic salts. The solution was washed with water, followed by washing with cold 0.5% aqueous NaOH (1 L) and then brine. The aqueous layers were back-extracted with MTBE (1 L). The combined organic layers were dried over sodium sulfate and concentrated. The final product could be isolated by nearly complete removal of MTBE followed by addition of cold hexane to precipitate the product from solution. The solid was then cold-filtered to give methyl 2-(3-bromo-5-formyl-4-methoxyphenyl)-2-methylpropanoate (130 g, 94%) as an off-white solid.

### Step 2

To a 5-L, 3-neck flask fitted with an addition funnel and mechanical stirrer was added 3-bromo-4-methoxybenzonitrile (Lancaster; 159.0 g; 750 mmol), anhydrous THF (3.0 L), and triisopropylborate (345 mL; 282 g; 1.50 mol). The solution was cooled to −78° C. in a dry ice/acetone bath then a solution of 2.44 M n-butyllithium in hexane (461 mL; 1.12 mol) was added over a 20-minute period. After the addition was complete, the reaction mixture was stirred at −78° C. for 1 hour. The reaction mixture was quenched with 7% aqueous phosphoric acid (2 L) and the reaction mixture was allowed to warm to room temperature. Stirring was stopped and the reaction mixture was allowed to stand overnight. The layers were separated, the aqueous phase discarded, and the organic phase was diluted with dichloromethane (2 L) and the organic phase was extracted with 5% aqueous sodium hydroxide (2×1.7 L). The aqueous phase was washed with MTBE (1.5 L) then acidified to pH=2.5 with 85% aqueous phosphoric acid, resulting in the formation of a white precipitate. The precipitate was filtered and washed with water to give 2-methoxy-5-cyanophenylboronic acid (104 g, 78%) as a white solid.

### Step 3

To a 5-L round-bottom flask with stir bar, heating mantle, reflux condenser, and thermometer was added methyl 2-(3-bromo-5-formyl-4-methoxyphenyl)-2-methyl-propanoate (114.5 g, 363 mmol), 2-methoxy-5-cyanophenylboronic acid (77.5 g, 438 mmol), THF (2.3 L), and N,N-diisopropylamine (169 mL; 1.21 mol). This solution was degassed at room temperature and PdCl₂(dppf).dichloromethane complex (3.4 g; 4.1 mmol) was added at room temperature. The temperature of the reaction mixture was increased to 70° C. and it was allowed to stir at that temperature for overnight. The next day, it was complete by HPLC analysis and the solution was allowed to cool to room temperature. The solvent was removed in vacuo. To this solution was added ethyl acetate (2 L) and the solution was extracted with a 5% solution of potassium carbonate in water (1.5 L) followed by an additional wash with brine (2.0 L). The organic layer was then treated with DARCO-60 charcoal (5.7 g) and this solution was allowed to stir at room temperature for 4 hours. The solution was then allowed to dry over sodium sulfate (200 g). The organic layer was then filtered through a fritted filter that was covered with celite (300 g), silica gel (300 g) and celite (300 g). The solids were washed with a 95:5 dichloromethane:methanol solution (1 L). The resulting solution was then concentrated to give methyl 2-(5′-cyano-5-formyl-6,2′-dimethoxybiphenyl-3-yl)-2-methylpropanoate (133 g) as an oil, which was taken onto the next step without any additional purification.

### Step 4

To a 3 L round bottom flask with a magnetic stir bar was added crude methyl 2-(5′-cyano-5-formyl-6,2′-dimethoxybiphenyl-3-yl)-2-methylpropanoate (133 g) and isopropanol (1.65 L). The solution was heated to 70° C. and a solution of sodium metabisulfite (69.0 g, 363 mmol) in water (650 mL) was added in one portion. The solution was allowed to stir at 70° C. for 1.5 hours then 3,4-diaminobenzamidine monohydrochloride (81.0 g, 434 mmol) was added. The solution was allowed to stir at 75-80° C. overnight while being left open to the air. The reaction mixture was concentrated at high temperature to remove about 75% of the isopropanol present and water (2.0 L) was added to the solution. The solution was cooled to 0° C. and filtered. The precipitate was washed with cold water (300 mL) and dried to yield methyl 2-[5-(5-carbamimidoyl-1H-benzoimidazol-2-yl)-5′-cyano-6,2′-dimethoxybiphenyl-3-yl]-2-methylpropanoate (155 g).

### Step 5

To a 12-L, 3-neck round bottom flask with a mechanical stirrer, a Dean-Stark condenser, heating mantle, and a nitrogen inlet was added pyridine hydrochloride (2.0 Kg, 17.31 mol) and toluene (1 L). The solution was heated to reflux overnight to remove 40 mL of excess water. The next day, methyl 2-[5-(5-carbamimidoyl-1H-benzoimidazol-2-yl)-5′-cyano-6,2′-dimethoxybiphenyl-3-yl]-2-methylpropanoate (155 g, 290 mmol) was added with toluene (500 mL). The internal temperature of the flask was raised to 175° C. where it then increased to 190° C. over 15 minutes with the temperature controller to the heating mantle having been turned off. The reaction was done after 0.5 h at 190° C. The stirring blade was removed from the solution and the melt was allowed to cool to room temperature where it solidified. To this solution water (8 L) was added and the solution was allowed to stir at room temperature overnight. The next morning the precipitated solid was removed from solution by filtration and it was washed with water (100 mL) to yield 2-[5-(5-carbamimidoyl-1H-benzoimidazol-2-yl)-5′-cyano-6,2′-dihydroxybiphenyl-3-yl]-2-methylpropanoic acid (135 g, 95%) that was greater than 98% pure by HPLC analysis.

### Step 6

2-[5-(5-Carbamimidoyl-1H-benzoimidazol-2-yl)-5′-cyano-6,2′-dihydroxy-biphenyl-3-yl]-2-methylpropanoic acid (0.762 g; 1.552 mmol) and HATU (0.768 g; 2.02 mmol) were dissolved in 10 mL of anhydrous N,N-dimethylacetamide. Pyridine (2.0 mL; 24.7 mmol) was added and the mixture was stirred for 1 h, then cooled to 0° C. Gaseous ammonia was then passed through the reaction mixture for 45 min. The reaction vessel was capped and the mixture stirred for 1 day at room temperature. The reaction mixture then was concentrated in vacuo and the residue was suspended in acetonitrile (40 mL) and sonicated for 20 min. The solid was filtered, washed with acetonitrile (20 mL) then dried in vacuo to give 2-[5-(5-carbamimidoyl-1H-benzoimidazol-2-yl)-6,2′-dihydroxy-5′-cyanobiphenyl-3-yl]isobutyramide hydrochloride as an off-white powder.

### Step 7

Palladium hydroxide on carbon (Pearlman's catalyst; 50% wet; 6.0 g) in trifluoroacetic acid (50 mL) was hydrogenated at 50 psi for 10 min. The crude 2-[5-(5-carbamimidoyl-H-benzoimidazol-2-yl)-6,2′-dihydroxy-5′-cyanobiphenyl-3-yl]isobutyramide hydrochloride was added to the reduced catalyst suspension, followed by additional amount of TFA (100 mL). The reaction mixture was hydrogenated at 50 psi until complete as determined by HPLC-UV analysis. A total reaction time of 5 hours was necessary. The reaction mixture was filtered through celite and the filtrated concentrated in vacuo. The residue was purified by preparative reverse phase HPLC using gradient elution with acetonitrile and water to give 2-[5′-aminomethyl-5-(5-carbamimidoyl-1H-benzoimidazol-2-yl)-6,2′-dihydroxybiphenyl-3-yl]isobutyramide dihydrochloride (457 mg, 55%) as a pale yellow powder.

### Step 8

Methyl (S)-(−)-2,2-dimethyl-1,3-dioxolane-4-carboxylate (5.33 g, 33.28 mmol; Aldrich catalogue number 25, 460-6) was dissolved in THF/water (1:1: 220 mL), containing an equimolar amount (1.40 g; 33.28 mmol) of LiOH monohydrate and stirred for 90 minutes at room temperature. The solution was concentrated in vacuo and dried to give (5.00 g, 99%) of the lithium salt (S)-(−)-2,2-dimethyl-1,3-dioxolane-4-carboxylic acid as a white solid. A portion of lithium salt (S)-(−)-2,2-dimethyl-1,3-dioxolane-4-carboxylic acid (105 mg; 0.65 mmol) and HATU (0.243 g; 0.64 mmol) in DMA (10 mL) were mixed and sonicated for 15 minutes until dissolution was achieved.

In a separate flask, 2-[5′-aminomethyl-5-(5-carbamimidoyl-1H-benzoimidazol-2-yl)-6,2′-dihydroxybiphenyl-3-yl]isobutyramide (0.31 g; 0.58 mmol), N,N-diisopropyl-ethylamine (0.113 mL; 0.65 mmol), pyridine (3.4 mL), and N,N-dimethylacetamide (15 mL) were stirred for 20 minutes until a clear solution was obtained. Both solutions were then combined and stirred for 3-4 h, using HPLC-UV analysis to monitor the progress of the reaction. After the reaction was determined to be complete, aqueous ammonium hydroxide (2 mL) was added and the mixture stirred for 4 h. The reaction mixture was concentrated under high vacuum and the residue suspended in acetonitrile (30 mL) then sonicated for 30 min. The precipitate was filtered, washed with acetonitrile and dried to afford N-[3′-(5-carbamimidoyl-1H-benzoimidazol-2-yl)-5′-(1-carbamoyl-1-methyl-ethyl)-6,2′-dihydroxybiphenyl-3-ylmethyl]-(S)-2,3-dihydroxypropionamide (440 mg) in 95% purity as determined by HPLC-UV analysis (AUC). This material was used directly in the next step.

### Step 9

N-[3′-(5-Carbamimidoyl-1H-benzoimidazol-2-yl)-5′-(1-carbamoyl-1-methylethyl)-6,2′-dihydroxybiphenyl-3-ylmethyl]-(S)-2,3-dihydroxypropionamide (0.41 g, 0.66 mmol) was dissolved 1 N aqueous HCl (5-6 mL) and stirred for 2 hours at room temperature. The solution was placed in a refrigerator for 1 h and the resulting crystalline product was isolated by filtration and washed twice with cold 1N HCl and redissolved in water (12 mL) and lyophilized to give the title compound (290 mg, 71%) as a light yellow solid in a purity of 97%, as determined by HPLC-UV and proton NMR analyses. Found (LCMS) 547.3 (M+1)⁺, 545.4 (M−1)⁻. Calc. for C₂₈H₃₀N₆O₆ 546.22.

### Example 2

### Synthesis of N-[3′-(5-carbamimidoyl-1H-benzoimidazol-2-yl)-5′-(1-carbamoyl-1-methyl-ethyl)-6,2′-dihydroxybiphenyl-3-ylmethyl]-(2S,3R)-2,3-dihydroxybutyramide

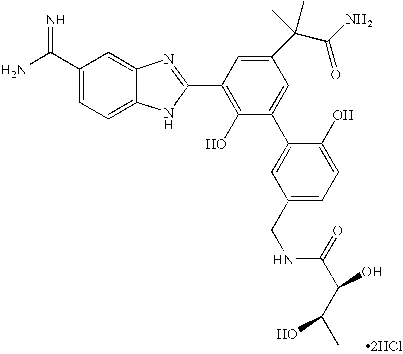

### Step 1

Methyl (2S,3R)-2,3-O-isopropylidene-2,3-dihydroxybutyrate (5.27 g, 30.25 mmol; Fluka catalogue number 59437) was dissolved in a solution of THF/water (1:1; 220 mL) containing an equimolar amount (1.27 g; 30.25 mmol) of lithium hydroxide monohydrate and stirred for 90 minutes. The solution was concentrated in vacuo to give the lithium salt (2S,3R)-2,3-O-isopropylidene-2,3-dihydroxybutyric acid (4.90 g, 98%) as a white solid. A portion of lithium salt (2S,3R)-2,3-O-isopropylidene-2,3-dihydroxybutyric acid and HATU (0.243 g; 0.64 mmol) in DMA (10 mL) were mixed then sonicated for 15 minutes until dissolution was achieved.

In a separate flask, a mixture of 2-[5′-aminomethyl-5-(5-carbamimidoyl-1H-benzoimidazol-2-yl)-6,2′-dihydroxybiphenyl-3-yl]isobutyramide hydrochloride (310 mg; 0.58 mmol), N,N-diisopropylamine (0.113 mL; 0.65 mmol), pyridine (3.4 mL) in DMA (15 mL) were stirred for 20 minutes until dissolution was complete. Both solutions were combined and the resulting reaction mixture was stirred for 3-4 hours. HPLC-UV analysis was employed to monitor the progress of the reaction. Once the reaction was deemed to be complete (4 h), aqueous ammonium hydroxide (2.0 mL) was added and the reaction mixture stirred for 4 h. The reaction mixture was concentrated under high vacuum and the residue suspended in acetonitrile (30 mL) and sonicated. The resulting precipitate was filtered, washed with acetonitrile and dried to give (4S,5R)-2,2,5-trimethyl-[1,3]dioxolane-4-carboxylic acid [3′-(5-carbamimidoyl-1H-benzoimidazol-2-yl)-5′-(1-carbamoyl-1-methylethyl)-6,2′-dihydroxybiphenyl-3-ylmethyl]amide hydrochloride (390 mg) which was 93% pure (AUC), as determined by HPLC-UV analysis.

### Step 2

The crude (4S,5R)-2,2,5-trimethyl-[1,3]dioxolane-4-carboxylic acid [3′-(5-carbamimidoyl-1H-benzoimidazol-2-yl)-5′-(1-carbamoyl-1-methylethyl)-6,2′-dihydroxybiphenyl-3-ylmethyl]amide (440 mg; 0.691 mmol) was dissolved in 1N HCl (5-6 mL) and stirred for 2 hours at room temperature. The reaction mixture was placed in a refrigerator and allowed to stand for 3 hours. The resulting precipitate was collected by filtration, washed with cold 1N aqueous HCl and dried in high vacuum overnight to give the title compound (250 mg, 57%), which was 97% pure as determined by HPLC-UV and proton NMR analyses. Found (LCMS) 561.3 (M+1)⁺, 559.4 (M−1)⁻. Calc. for C₂₉H₃₂N₆O₆ 560.24

### Example 3

### Synthesis of 2S-{2-[5-(5-carbamimidoyl-1H-benzoimidazol-2-yl)-6,2′-dihydroxy-5′-sulfamoylbiphenyl-3-yl]-2-methylpropionylamino} succinamide

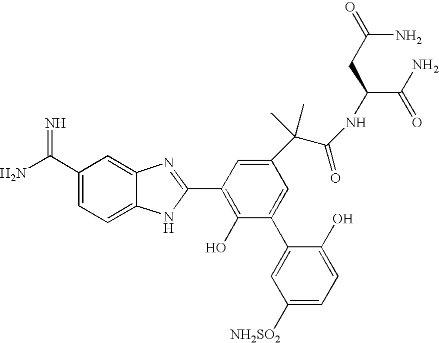

### Step 1

To a solution of 2-methoxy-5-tert-butylsulfamoylphenylboronic acid (4.08 g, 14.28 mmol) was dissolved in methanol (36 mL) was added 2-(3-bromo-5-formyl-4-methoxyphenyl)-2-methylpropionate (3.0 g, 9.50 mmol) and toluene (90 mL). Potassium carbonate solution (7.14 mL, 2 M, 14.28 mmol) was added and the reaction mixture was flushed with nitrogen. Tetrakis(triphenylphosphine)palladium (1.10 g, 0.95 mmol) was added and the reaction mixture is refluxed for 3 hours. After cooling, the reaction mixture is partitioned with 5% citric acid solution and the organic phase is dried and evaporated. Purification by column chromatography (40% EtOAc/hexane) provided methyl 2-(5′-tert-butylsulfamoyl-5-formyl-6,2′-dimethoxybiphenyl-3-yl)-2-methyl-propionate (3.79 g, 84%).

### Step 2

Methyl 2-(5′-tert-butylsulfamoyl-5-formyl-6,2′-dimethoxybiphenyl-3-yl)-2-methylpropionate (3.79 g, 7.94 mmol) was dissolved in methanol (150 mL) and 3,4-diaminobenzamidine HCl (1.35 g, 7.25 mmol) and p-benzoquinone (0.78 g) were added and the reaction mixture was refluxed overnight. The reaction mixture was cooled and evaporated to dryness to give methyl 2-[5-(5-carbamimidoyl-1H-benzoimidazol-2-yl)-6,2′-dihydroxy-5′-tert-butylsulfamoylbiphenyl-3-yl]-2-methylpropionate which was dissolved in trifluoroacetic acid (25 mL) and stirred for an hour. The volatiles are evaporated to give crude methyl 2-[5-(5-carbamimidoyl-1H-benzoimidazol-2-yl)-6,2′-dihydroxy-5′-sulfamoylbiphenyl-3-yl]-2-methylpropionate. To the crude methyl 2-[5-(5-carbamimidoyl-1H-benzoimidazol-2-yl)-6,2′-dihydroxy-5′-sulfamoylbiphenyl-3-yl]-2-methylpropionate was added pyridine-HCl (20 g) and the mixture was heated at 180° C. for 3 hours. After cooling, the solid is dissolved in 5% MeCN/water and purified by preparative HPLC and the eluent containing the product was lyophilized to give 2-[5-(5-carbamimidoyl-1H-benzoimidazol-2-yl)-6,2′-dihydroxy-5′-sulfamoylbiphenyl-3-yl]-2-methylpropionic acid (3.57 g, 82%).

### Step 3

2-[5-(5-Carbamimidoyl-1H-benzoimidazol-2-yl)-6,2′-dihydroxy-5′-sulfamoyl-biphenyl-3-yl]-2-methylpropionic acid (250 mg, 0.458 mmol) was dissolved in DMA (100 mL) and the solution is charged with HATU (192 mg, 0.504 mmol) and collidine (243 uL, 1.83 mmol) and stirred for two hours. Asparagine amide-HCl (0.154 g, 0.916 mmol) was added with TEA (139 uL). The reaction mixture was stirred overnight and the pH was adjusted to about 3 and the solvents are evaporated. This crude was purified by preparative HPLC and the fractions containing the product were lyophilized to give the title compound (228 mg, 66%). LCMS, Calcd=622.65; Obsvd (MH⁺)=623.3, (MH—)=621.2. NMR (400 MHz) (DMSO-d₆) δ 1.62 (s, 6H), 2.68 (m, 1H), 3.10 (m, 1H), 3.60 (m, 1H), 4.57 (m, 4H), 7.08 (d, J=5 Hz, 1H) 7.18 (br.s, 2H), 7.35 (d, J=1.5 Hz, 1H), 7.42 (br.s, 1H), 7.67 (m, 2H), 7.76 (dd, J=1.5 Hz, 1H), 7.88 (d, J=5 Hz, 1H), 8.18 (br. s, 1H), 8.21 (d, J=1.5 Hz, 1H), 9.12, 9.43 (2s, 4H).

Proceeding as described above but substituting asparigine amide-HCl with N-methyl-D-glucamine provided 2-[5-(5-carbamimidoyl-1H-benzoimidazol-2-yl)-6,2′-dihydroxy-5-sulfamoylbi-phenyl-3-yl]-N-methyl-N-(2R,3S,4S,5S,6-pentahydroxyhexyl)-isobutyramide,

LCMS, Calcd=686.73; Obsvd (MH⁺)=687.5, (MH—)=685.4. NMR (400 MHz) (DMSO-d₆) δ 1.60 (4s, 6H), 2.65 (s, 3H), 3.20-4.10 (m, 13H), 7.08 (d, J=5 Hz, 1H), 7.18 (br. s, 2H), 7.41 (d, J=1.5 Hz, 1H), 7.68 (m, 2H), 7.82 (dd, J=1.5 Hz, 1H), 8.03 (d, J=5 Hz, 1H), 8.20 (br.s, 1H), 8.21 (d, J=1.5 Hz, 1H), 8.95, 9.18 (2s, 4H), 10.35 (s, 1H).

### Example 4

### Synthesis of 2-{5-(5-carbamimidoyl-1H-benzoimidazol-2-yl)-6,2′-dihydroxy-5′-[(2S-hydroxypropionylamino)methyl] biphenyl-3-yl} isobutyramide dihydrochloride

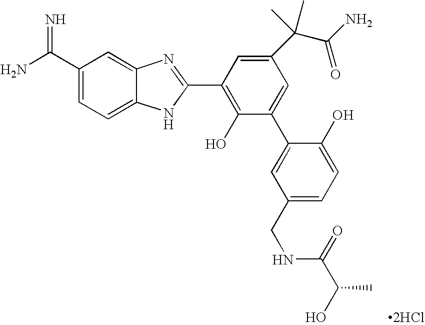

### Step 1

Diisopropylethylamine (3.5 mL, 20 mmol) was added to suspension of 4-nitrophenol (2.78 g, 20 mmol) in dichloromethane (60 mL). The solution was cooled to −20° C. and a solution of (S)-2-acetoxypropionyl chloride (3.01 g, 20 mmol) dichloromethane (12 mL) was added dropwise over 15 min. The reaction mixture was stirred at this temperature for 3 hours and then poured into 0.5 N aqueous HCl (300 mL). Organic layer was diluted by dichloromethane (100 mL), washed with water, brine and dried over magnesium sulfate. After evaporation of solvents (S)-2-acetoxypropionic acid 4-nitro-phenyl ester (5.08 g, 00%) was obtained.

### Step 2

A solution of (S)-2-acetoxypropionic acid 4-nitrophenyl ester (0.046 g, 0.18 mmol) in dimethylacetamide (1 mL) was added to a mixture of 2-[5′-aminomethyl-5-(5-carbamimidoyl-1H-benzoimidazol-2-yl)-6,2′-dihydroxy-biphenyl-3-yl]-isobutyramide dihydrochloride (0.0905 g, 0.17 mmol) and triethylamine (0.05 mL, 0.357 mmol) in dimethylacetamide (3 mL). The reaction mixture was stirred for 3 hours, quenched by dropwise addition of 3 ml of conc. aqueous ammonia and left for 14 hours. Solvents were rotoevaporated in high vacuum and the residue re-dissolved in water. Purification by RP-HPLC (acetonitrile gradient) to give the title compound (0.08 g, 78%) as a pale yellow amorphous solid after lyophilization. Found (LCMS) 531.3 (M+1)⁺, 529.4 (M−1)⁻. Calc. for C₂₉H₃₀N₆O₅ 530.23

### Example 1

### In Vitro Factor VIIa Inhibitor Assay

Mixtures of human Factor VIIa (typically supplied at 7 nM) and test compound (present at varying concentrations) in assay medium (comprising: NaCl, 150 mM (pH 7.4); CaCl₂, 5 mM; Tween-20, 0.05%; Dade Innovin tissue factor [Dade Behring, Newark, Del., USA]; EDTA, 1.5 mM; and dimethylsulfoxide, 10%) were incubated for 30 minutes at room temperature. Next, reactions were initiated with the addition of substrate [500 μM of CH₃SO₂-D-Cha-But-Arg-pNA (from Centerchem, Norwalk, Conn., USA)]. Hydrolysis of the chromogenic substrate was followed spectrophotometrically at 405 nm for five minutes. Initial velocity measurements calculated from the progress curves by a kinetic analysis program (Batch Ki; BioKin, Ltd., Pullman, Wash.) were used to determine apparent inhibition constants (apparent Kᵢ's).

Compounds of the invention tested by the above-described assay exhibited inhibition of Factor VIIa.

### Example 2

### In Vitro Factor Xa Inhibitor Assay

Mixtures of human Factor Xa (typically supplied at 3 nM) (from Haematologic Technologies, Essex Junction, Vt., USA) and test compound (varying concentrations) in assay medium (comprising: Tris, 50 mM (pH 7.4); NaCl, 150 mM; CaCl₂, 5 mM; Tween-20, 0.05%; EDTA, 1 mM; and dimethylsulfoxide, 10%) were incubated for 30 minutes at room temperature. Next, reactions were initiated with the addition of substrate [500 μM of CH₃CO₂-D-Cha-Gly-Arg-pNA (from Centerchem, Norwalk, Conn., USA]. Hydrolysis of the chromogenic substrate was followed spectrophotometrically at (405 nm) for five minutes. Apparent inhibition constants (apparent Kᵢ's) were calculated from the enzyme progress curves using standard mathematical models.

Compounds of the invention tested by the above-described assay exhibited inhibition of Factor Xa.

### Example 3

### Pharmacokinetic Assay

Rats with pre-implanted jugular vein catheters, which were filled with heparin/saline/PVP lock prior to shipment, were bought from Charles River. Three rats were selected for each study, weighed, and injected with test compound by tail vein injection. Any residual test compound was retained and stored at −70° C. for later analysis.

Blood samples (0.25 mL each) were collected from the indwelling catheters at specified times over 120 h. The catheters were flushed with physiological saline immediately after each collection and filled with heparinized saline after each 8, 24 and 48 h collection. In the event that a catheter failed, blood samples were collected via the retro-orbital sinus under isoflurane anesthesia at the appropriate time.

Blood samples were placed in 0.5 mL Microtainer® tubes (lithium heparin), shaken gently and stored on wet ice. The samples were centrifuged for 10 minutes at 2400 rpm in a refrigerated centrifuged. Plasma samples (0.1 mL) from each tube were transferred to 0.5 mL Unison polypropylene vials (Sun—500210) and stored below −70° C. for later analysis by LC/MS-MS.

### Example 4

### In vitro Clotting Assays . . . aPTT and PT

Coagulation assays, activated partial thromboplastin time (aPTT) and prothrombin time (PT) were carried out based on the procedure described in Hougie, C. Hematology (Williams, W. J., Beutler, B., Erslev, A. J., and Lichtman, M. A., Eds.), pp. 1766-1770 (1990), McGraw-Hill, New York.

Briefly, the assays were performed using normal human citrated plasma and were performed at 37° C. on a coagulometer (Electra 800) in accordance with the manufacturer's instructions (Medical Laboratory Automation-Pleasantville, N.Y.). The instrument was calibrated with plasma immediately prior to collecting clotting times for samples with inhibitors. The aPTT and PT doubling concentrations were calculated by fitting inhibitor dose response curves to a modified version of the Hill equation.

### Pharmaceutical Composition Examples

The following are representative pharmaceutical formulations containing a compound of this invention.

Tablet Formulation
The following ingredients are mixed intimately
and pressed into single scored tablets.

| Ingredient | Quantity per tablet, mg |
|---|---|
| compound of this invention | 400 |
| cornstarch | 50 |
| croscarmellose sodium | 25 |
| lactose | 120 |
| magnesium stearate | 5 |

Capsule Formulation
The following ingredients are mixed intimately
and loaded into a hard-shell gelatin capsule.

| Ingredient | Quantity per capsule, mg |
|---|---|
| compound of this invention | 200 |
| lactose, spray-dried | 148 |
| magnesium stearate | 2 |

Suspension Formulation
The following ingredients are mixed to form
a suspension for oral administration.

| Ingredient | Amount | |
|---|---|---|
| compound of this invention | 1.0 | g |
| fumaric acid | 0.5 | g |
| sodium chloride | 2.0 | g |
| methyl paraben | 0.15 | g |
| propyl paraben | 0.05 | g |
| granulated sugar | 25.5 | g |
| sorbitol (70% solution) | 12.85 | g |
| Veegum K (Vanderbilt Co.) | 1.0 | g |
| flavoring | 0.035 | mL |
| colorings | 0.5 | mg |
| distilled water | q.s. to 100 | mL |

Injectable Formulation
The following ingredients are mixed
to form an injectable formulation.

| Ingredient | Amount | |
|---|---|---|
| compound of this invention | 1.2 | g |
| sodium acetate buffer solution, | 0.4 M 2.0 | mL |
| HCl (1 N) or NaOH (1 N) | q.s. to suitable | pH |
| water (distilled, sterile) | q.s. to 20 | mL |

All of the above ingredients, except water, are combined and heated to
60-70° C. with stirring. A sufficient quantity of water at 60° C.
is then added with vigorous stirring to emulsify the ingredients, and
water then added q.s. to 100 g.

Suppository Formulation
A suppository of total weight 2.5 g is prepared by mixing the compound
of the invention with Witepsol ® H-15 (triglycerides of saturated
vegetable fatty acid; Riches-Nelson, Inc., New York), and has the
following composition:

| | | |
|---|---|---|
| compound of the invention | 500 | mg |
| Witepsol ® H-15 | balance | |

Parenteral Formulation

| | | |
|---|---|---|
| Compound of this invention | 40 | mg/mL |
| Hydroxypropyl-β-cyclodextrin | 200 | mg/mL |

Adjust pH with 1.0 N sodium hydroxide to 7.4

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

We claim:

1. A compound selected from the group consisting of consisting of compounds (a)-(k): 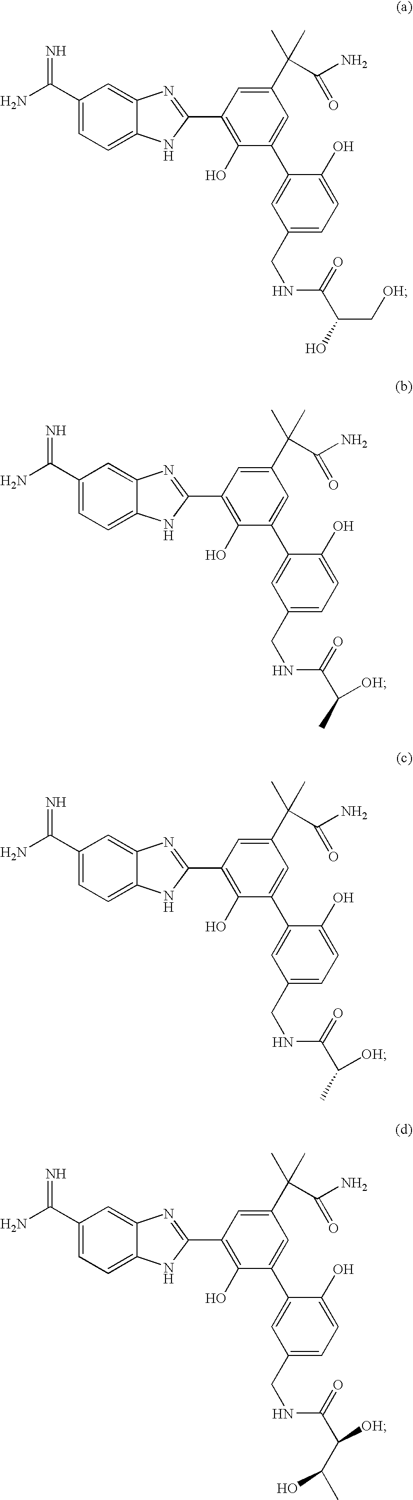 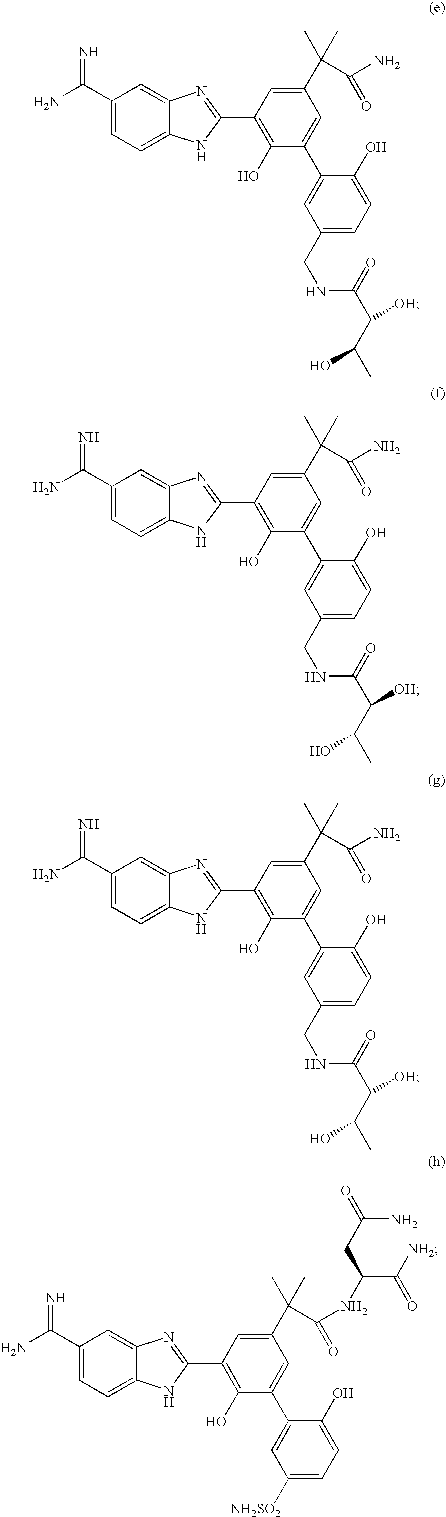 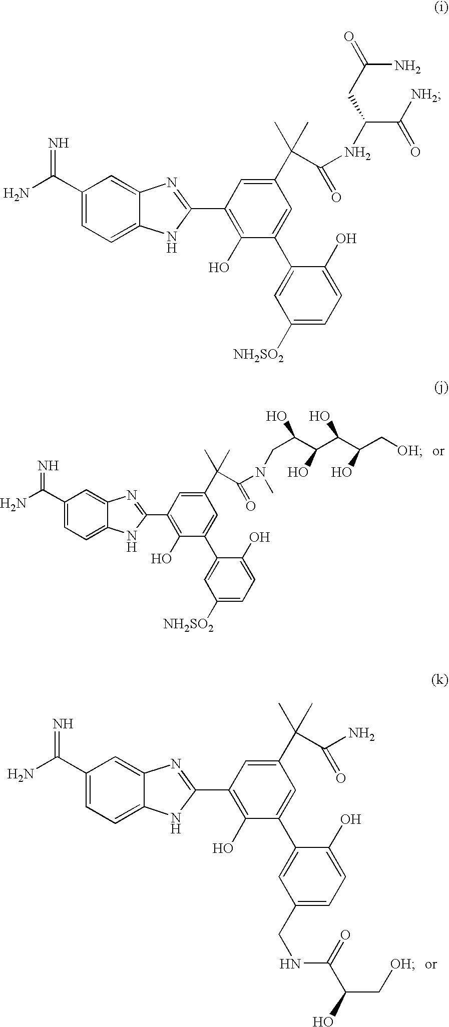
a pharmaceutically acceptable salt thereof.

2. A pharmaceutical composition comprising a pharmaceutically acceptable carrier and a therapeutically effective amount of compound (a), (b), (c), (d), (e), (f), (g), (h), (i), (j), or (k); or a pharmaceutically acceptable salt thereof.

3. A method of treating a disease in an animal that is mediated by Factors VIIa, IXa, Xa and/or XIa which method comprises administering to said animal a therapeutically effective amount of compound (a), (b), (c), (d), (e), (f), (g), (h), (i), (O), or (k); or a pharmaceutically acceptable salt thereof.

4. The method of claim 3 wherein the disease is mediated by Factor VIIa.

5. A method of treating a thromboembolic disorder in an animal which method comprises administering to said animal a therapeutically effective amount of compound (a), (b), (c), (d), (e), (f), (g), (h), (i), (O), or (k); or a pharmaceutically acceptable salt thereof in combination with another anticoagulant agent(s) independently selected from a group consisting of a thrombin inhibitor, factor IXa inhibitor, factor Xa inhibitor, Aspirin®, and Plavix®.
